(19) United States
(12) Patent Application Publication
(10) Pub. No.: US 2023/0125578 A1
(43) Pub. Date: Apr. 27, 2023

(54) MEDICAL FIXING SYSTEM

(71) Applicants: Chia-Ming Lu, New Taipei City (TW); Cheng-Hung Lin, New Taipei City (TW); Janus Biomedical International Co., Ltd., New Taipei City (TW)

(72) Inventors: Chia-Ming Lu, New Taipei City (TW); Cheng-Hung Lin, New Taipei City (TW)

(21) Appl. No.: 17/877,694

(22) Filed: Jul. 29, 2022

(30) Foreign Application Priority Data
Oct. 22, 2021 (TW) 110139174

(51) Int. Cl. A61B 17/08

(52) U.S. Cl. CPC A61B 17/085; A61B 2017/086; A61B 2090/064

(57) ABSTRACT

A medical fixing system includes: a first fixing device fixed to a target lesion and having a first joining part and a first auxiliary fixing portion; a second fixing device fixed to the target lesion and having a second auxiliary fixing portion; a cord-receiving device connected to the first joining part of the first fixing device; and a fixing cord in contact with the first auxiliary fixing portion and the second auxiliary fixing portion and connected to the cord-receiving device. The cord-receiving device has a cord-receiving mechanism. The cord-receiving device adjusts an exposed length of the fixing cord with the cord-receiving mechanism.

### BACKGROUND OF THE INVENTION

### Field of the Invention

The present disclosure relates to medical fixing systems and, more particularly, to a medical fixing system conducive to drawing the skin surrounding a large-area wound or lesion closer thereto and thereby speeding up the healing of the wound or lesion.

### Description of the Prior Art

Conventional medical fixing systems help draw the skin surrounding a wound or lesion closer thereto. However, during the period of time when the conventional medical fixing systems are mounted in place at the wound or lesion, the surrounding skin is likely to shift toward or away from the wound or lesion for various reasons (for example, the patient's body movement or inflammation of the wound). Being incapable of adjusting the magnitude of a pull they exert on the wound or lesion, the conventional medical fixing systems are likely to exert a pull whose magnitude decreases greatly and thereby reduces skin drawing efficacy or increases greatly and thereby exacerbates the wound or lesion. In particular, when the wound or lesion is close to the thoracic cavity, or after the patient has undergone thoracic surgery, the wound is often pulled instantaneously due to the expansion and contraction of the thoracic cavity (for example, caused by the patient's breathing or coughing), which makes the healing of the wound more difficult. As a result, healthcare workers have to mount the medical fixing systems in place anew, not only taking much time, but also causing secondary injury to the wound or lesion. Therefore, it is necessary to provide a medical fixing system capable of adjusting the magnitude of a pull it exerts on a wound or lesion in real time and as needed.

### SUMMARY OF THE INVENTION

In view of the aforesaid drawbacks of the prior art, it is an objective of the present disclosure to provide a medical fixing system capable of adjusting the magnitude of a pull it exerts on a wound or lesion in real time and as needed.

It is another objective of the present disclosure to provide a medical fixing system capable of effectively reducing the pressure on the skin surface of a wound or lesion and reducing excessive and instantaneous strain injuries.

In order to achieve the above and other objectives, the present disclosure provides a medical fixing system, comprising: a first fixing device fixed to a target lesion and having a first joining part and a first auxiliary fixing portion; a second fixing device fixed to the target lesion and having a second auxiliary fixing portion; a cord-receiving device connected to the first joining part of the first fixing device; and a fixing cord in contact with the first auxiliary fixing portion and the second auxiliary fixing portion and connected to the cord-receiving device, wherein the cord-receiving device has a cord-receiving mechanism and adjusts an exposed length of the fixing cord with the cord-receiving mechanism.

In a preferred embodiment of the present disclosure, the first fixing device has a first adhesive side and is fixed to the target lesion through the first adhesive side, and the second fixing device has a second adhesive side and is fixed to the target lesion through the second adhesive side.

In a preferred embodiment of the present disclosure, the cord-receiving device comprises: a first cord-receiving portion having a second joining part and connected to the first joining part of the first fixing device by the second joining part; and a second cord-receiving portion, with the fixing cord being fixed to the second cord-receiving portion, wherein the cord-receiving mechanism is the second cord-receiving portion's being pivotally rotatable relative to the first cord-receiving portion while being connected to the first cord-receiving portion.

In a preferred embodiment of the present disclosure, the exposed length of the fixing cord increases gradually when the second cord-receiving portion rotates pivotally in a first direction relative to the first cord-receiving portion, wherein the exposed length of the fixing cord decreases gradually when the second cord-receiving portion rotates pivotally in a second direction relative to the first cord-receiving portion.

In a preferred embodiment of the present disclosure, the first joining part is a dovetail male socket, and the second joining part is a dovetail sockets.

In a preferred embodiment of the present disclosure, the second cord-receiving portion has a third auxiliary fixing portion, and the fixing cord is fixed to the third auxiliary fixing portion of the cord-receiving device.

In a preferred embodiment of the present disclosure, the first cord-receiving portion has a first engaging portion, and the second cord-receiving portion has a second engaging portion, wherein the first engaging portion and the second engaging portion are engaged with each other when the cord-receiving device is in a first state, allowing the first cord-receiving portion to be fixed to the second cord-receiving portion, wherein the first engaging portion and the second engaging portion are separated from each other when the cord-receiving device is in a second state, allowing the second cord-receiving portion to rotate pivotally relative to the first cord-receiving portion.

In a preferred embodiment of the present disclosure, the cord-receiving device comprises a resilient mountings disposed between the first cord-receiving portion and the second cord-receiving portion and in contact with the first cord-receiving portion and the second cord-receiving portion, wherein the cord-receiving device is in the first state when the resilient mountings is in a stretched state, wherein the cord-receiving device is in the second state when the resilient mountings is in a compressed state.

In a preferred embodiment of the present disclosure, the cord-receiving device comprises a first sensing portion and an warning signal, wherein the first sensing portion senses a tension of the fixing cord and generates an data alert when the tension is less than a first tension threshold or greater than a second tension threshold, wherein the warning signal sends an warning signal according to the data alert.

In a preferred embodiment of the present disclosure, the first sensing portion generates a tension data according to the tension of the fixing cord and sends the data alert and/or the tension data to a data-receiving device when the tension is less than the first tension threshold or greater than the second tension threshold.

In a preferred embodiment of the present disclosure, the cord-receiving device comprises a second sensing portion and an warning signal, wherein the second sensing portion senses a distance between the cord-receiving device and the second fixing device and generates an data alert when the distance is less than a first distance threshold or greater than a second distance threshold, wherein the warning signal sends an warning signal according to the data alert.

In a preferred embodiment of the present disclosure, the second sensing portion generates a distance data according to the distance between the cord-receiving device and the second fixing device and sends the data alert and/or the distance data to a data-receiving device when the distance is less than the first distance threshold or greater than the second distance threshold.

In order to achieve the above and other objectives, the present disclosure further provides a medical fixing system, capable of adjusting an exposed length of a fixing cord at a target lesion, the medical fixing system comprising: a first fixing device fixed to the target lesion and having a first joining part and a first auxiliary fixing portion; a second fixing device fixed to the target lesion and having a second auxiliary fixing portion; and a cord-receiving device connected to the first joining part of the first fixing device, wherein the fixing cord is connected to the cord-receiving device and in contact with the first auxiliary fixing portion and the second auxiliary fixing portion, wherein the cord-receiving device has a cord-receiving mechanism and adjusts the exposed length of the fixing cord with the cord-receiving mechanism to not only allow the fixing cord to drive the first fixing device and the second fixing device but also allow the first fixing device and the second fixing device to be drawn closer to or away from each other.

In a preferred embodiment of the present disclosure, the cord-receiving device comprises: a first cord-receiving portion having a second joining part and connected to the first joining part of the first fixing device by the second joining part; and a second cord-receiving portion, with the fixing cord being fixed to the second cord-receiving portion, wherein the cord-receiving mechanism is the second cord-receiving portion's being pivotally rotatable relative to the first cord-receiving portion while being connected to the first cord-receiving portion.

In a preferred embodiment of the present disclosure, the first cord-receiving portion has a first engaging portion, and the second cord-receiving portion has a second engaging portion, wherein the first engaging portion and the second engaging portion are engaged with each other when the cord-receiving device is in a first state, allowing the first cord-receiving portion to be fixed to the second cord-receiving portion, wherein the first engaging portion and the second engaging portion are separated from each other when the cord-receiving device is in a second state, allowing the second cord-receiving portion to rotate pivotally relative to the first cord-receiving portion.

In a preferred embodiment of the present disclosure, the cord-receiving device comprises a resilient mountings disposed between the first cord-receiving portion and the second cord-receiving portion and adapted to be in contact with the first cord-receiving portion and the second cord-receiving portion, wherein the cord-receiving device is in the first state when the resilient mountings is in a stretched state, wherein the cord-receiving device is in the second state when the resilient mountings is in a compressed state.

The aforesaid aspects and other aspects of the present disclosure are described in detail in accordance with the non-restrictive, specific embodiments below and depicted with the accompanying drawings.

### BRIEF DESCRIPTION OF THE DRAWINGS

FIG. 1A is a schematic view of a medical fixing system according to a specific embodiment of the present disclosure.

FIG. 1B is a schematic view of the medical fixing system according to a specific embodiment of the present disclosure.

FIG. 2A is a schematic view of a first fixing device according to a specific embodiment of the present disclosure.

FIG. 2B is a schematic view of the first fixing device according to a specific embodiment of the present disclosure.

FIG. 3A is a schematic view of a cord-receiving device according to a specific embodiment of the present disclosure.

FIG. 3B is a schematic view of the cord-receiving device according to a specific embodiment of the present disclosure.

FIG. 4 is a schematic view of a cord-receiving device connected to the first fixing device according to a specific embodiment of the present disclosure.

FIG. 5 is a schematic view of a cord-receiving device according to a specific embodiment of the present disclosure.

FIG. 6A is a schematic view of a cord-receiving device according to a specific embodiment of the present disclosure.

FIG. 6B is a schematic view of the cord-receiving device according to a specific embodiment of the present disclosure.

### DETAILED DESCRIPTION OF THE EMBODIMENTS

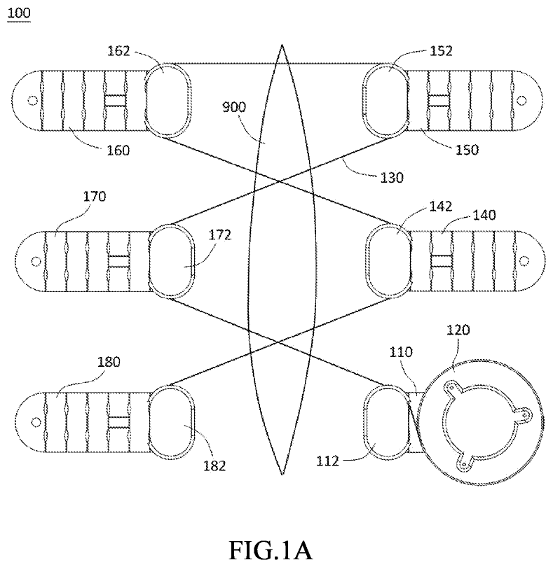

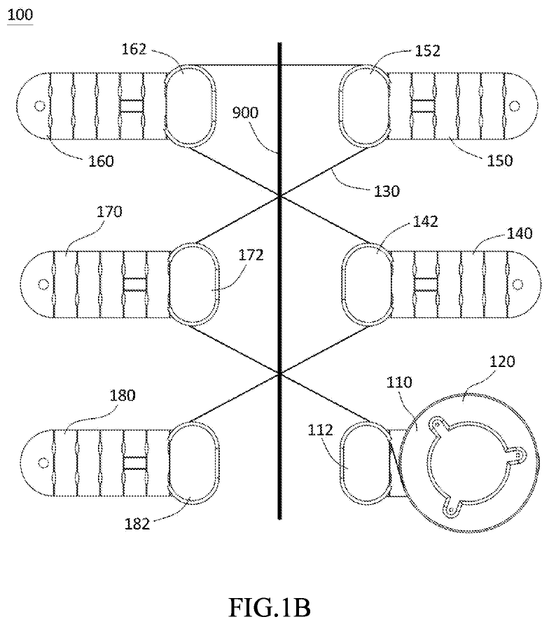

Referring to FIG. 1A and FIG. 1B, there are shown schematic views of a medical fixing system according to a specific embodiment of the present disclosure. In the embodiment illustrated by FIG. 1A and FIG. 1B, a medical fixing system 100 adjusts the exposed length of a fixing cord 130 at a target lesion 900 (the target lesion 900 is, for example, a large-area wound, but the present disclosure is not limited thereto,) such that the skin surrounding the target lesion 900 stays at an appropriate position. For example, the skin surrounding the target lesion is maintained in a closed-up state. The medical fixing system 100 comprises a first fixing device 110, cord-receiving device 120 and second fixing devices 140, 150, 160, 170, 180. The first fixing device 110 and second fixing devices 140, 150, 160, 170, 180 are fixed to the target lesion 900. Preferably, the first fixing device 110 and second fixing devices 140, 150, 160, 170, 180 are fixed to the skin surrounding the target lesion 900. The cord-receiving device 120 is connected to the first fixing device 110. The first fixing device 110 has a first auxiliary fixing portion 112. The second fixing devices 140, 150, 160, 170, 180 have second auxiliary fixing portions 142, 152, 162, 172, 182, respectively. The fixing cord 130 is in contact with the second auxiliary fixing portions 142, 152, 162, 172, 182 and first auxiliary fixing portion 112, because, for example, the fixing cord winds around or surrounds at least part of the second auxiliary fixing portions, but the present disclosure is not limited thereto. The fixing cord 130 is connected to the cord-receiving device 120; for example, the fixing cord 130 is fixed to the cord-receiving device 120. In different specific embodiments, the fixing cord 130 is regarded as one of the elements of the medical fixing system 100 as needed, or the fixing cord 130 is not regarded as one of the elements of the medical fixing system 100 as needed. In one embodiment, the fixing cord 130 is selected from medical sutures, and elastic bands or wires. When the fixing cord 130 is an elastic band or wire (such as a plastic band or wire of elasticity or flexibility), the slight stretchability of the elastic band or wire may offset part of the instantaneous tension, and reduce the pressure on the skin surface of the wound or lesion, thus reducing excessive strain injuries. In addition, the elastic band or wire can massage the skin of the wound or lesion in response to the natural movement of the skin of the wound or lesion (for example, caused by the patient's breathing), which is helpful for the healing and recovery of the wound.

In the embodiment illustrated by FIG. 1A and FIG. 1B, the cord-receiving device 120 has a cord-receiving mechanism. The cord-receiving device 120 adjusts the exposed length of the fixing cord 130 and the tightness of the fixing cord 130 (i.e., the tension of the fixing cord 130) with the cord-receiving mechanism. For example, in the embodiment illustrated by FIG. 1A, the cord-receiving device 120 uses the cord-receiving mechanism to extend the exposed length of the fixing cord 130, so as to not only drive the first fixing device 110 and second fixing devices 140, 150, 160, 170, 180, but also move the first fixing device 110 and the second fixing devices 140, 150, 160, 170, 180 away from each other, thereby enlarging the target lesion 900. In the embodiment illustrated by FIG. 1B, the cord-receiving device 120 uses the cord-receiving mechanism to reduce the exposed length of the fixing cord 130, so as to not only drive the first fixing device 110 and second fixing devices 140, 150, 160, 170, 180, but also draw the first fixing device 110 and second fixing devices 140, 150, 160, 170, 180 closer to each other, thereby diminishing the target lesion 900. The medical fixing system 100 uses the cord-receiving mechanism of the cord-receiving device 120 to adjust the exposed length of the fixing cord 130 and the tightness of the fixing cord 130 in real time, so as to drive the first fixing device 110 and second fixing devices 140, 150, 160, 170, 180 and adjust their distances or positions.

Conventional medical fixing systems do not use a fixing cord to draw fixing devices closer to each other; instead, conventional medical fixing systems use a cable tie to fix paired fixing devices to each other. Thus, the conventional fixing devices are able to adjust the distance between a pair of fixing devices at most but not simultaneously the positions of all the fixing devices. Furthermore, the fixing devices of the conventional medical fixing systems have to be symmetrically arranged, and thus the total number of the fixing devices must be an even number. By contrast, the first fixing device and the second fixing devices of the medical fixing system of the present disclosure need not be symmetrically arranged, and their total number is not necessarily an even number but may also be an odd number, for example, 5, 7 or 9, as needed; however, the present disclosure is not limited thereto. Furthermore, the medical fixing system of the present disclosure adjusts the positions of all the fixing devices and the tightness of a fixing cord and thus demonstrates a high degree of flexibility and adaptivity. The medical fixing system of the present disclosure is not only applicable to linear wounds but also applicable to wounds of different shapes, for example, irregular shapes such as roundish shape and irregular polygonal shape, as needed, but the present disclosure is not limited thereto.

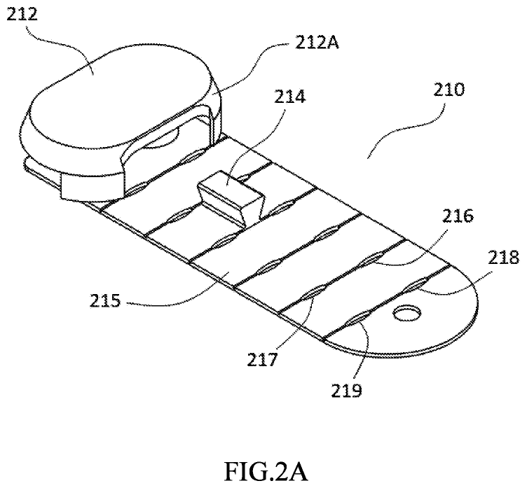

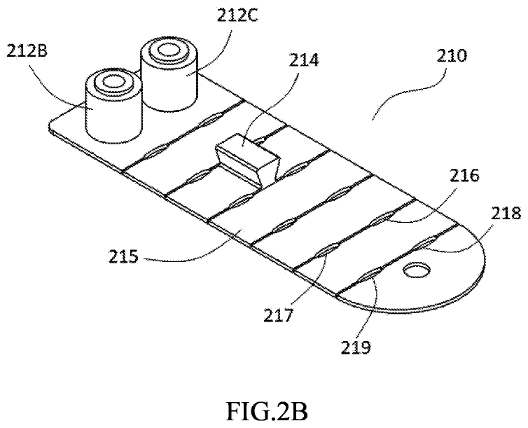

Referring to FIG. 2A and FIG. 2B, there are shown schematic views of a first fixing device according to a specific embodiment of the present disclosure. FIG. 2B does not show a cover portion 212A of FIG. 2A. In the embodiment illustrated by FIG. 2A and FIG. 2B, a first fixing device 210 has a first body portion 215, first joining part 214 and first auxiliary fixing portion 212. The first body portion 215 has a first side facing the target lesion and a second side facing away from the first side. The first joining part 214 and the first auxiliary fixing portion 212 are disposed on and connected to the first body portion 215. Preferably, the first joining part 214 and the first auxiliary fixing portion 212 are disposed on the second side of the first body portion 215. Preferably, the first side of the first body portion 215 is a first adhesive side, and the first fixing device 210 is fixed to the target lesion through the first adhesive side. The first joining part 214 is connected to the first joining part 214. In a specific embodiment, the first joining part 214 is a dovetail male socket.

In the embodiment illustrated by FIG. 2A and FIG. 2B, the first auxiliary fixing portion 212 has a cover portion 212A and cord contact portions 212B, 212C. The cord contact portions 212B, 212C are disposed between the first body portion 215 and the cover portion 212A. In the process of winding a fixing cord around each fixing device, the fixing cord is in contact with at least one of the cord contact portions 212B, 212C; for example, the fixing cord winds around or surrounds at least part of the cord contact portions, but the present disclosure is not limited thereto. Thus, the fixing cord is movably fixed in place between the fixing devices. The cover portion 212A blocks the fixing cord and keeps the fixing cord between the first body portion 215 and the cover portion 212A to prevent the fixing cord from sliding and thus preclude its resultant complete separation from the first fixing device 210.

The first body portion 215 has through holes 216, 217, 218, 219. Healthcare workers fix the first fixing device 210 to the target lesion (with, for example, sutures or staples, but the present disclosure is not limited thereto) through the through holes 216, 217, 218, 219 as needed. In this regard, healthcare workers do not necessarily fix the first fixing device 210 to the target lesion by one single technique; instead, healthcare workers may fix the first fixing device 210 to the target lesion by one or more techniques at the same time as needed; for example, healthcare workers may fix the first fixing device 210 to the target lesion with adhesives and sutures at the same time.

In a specific embodiment, the second fixing device and the first fixing device are identical in terms of structures and features, and the second fixing device is fixed to the target lesion in the same way as the first fixing device to the target lesion. Thus, healthcare workers may connect a cord-receiving device to a joining part of any second fixing device as needed (such that the second fixing device connected to the cord-receiving device can be regarded as the first fixing device.) In another specific embodiment, the second fixing device lacks a first joining part but is exactly identical to the first fixing device in terms of structures, features and/or the way of being fixed to the target lesion. For example, in a specific embodiment, the second fixing device has a second adhesive side, and the second fixing device is fixed to the target lesion through the second adhesive side.

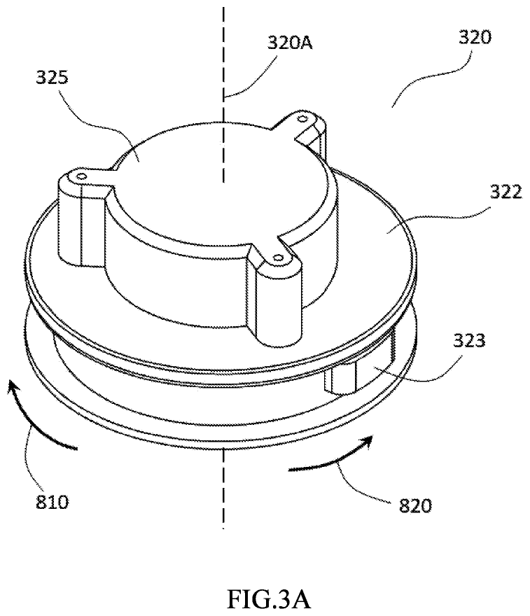

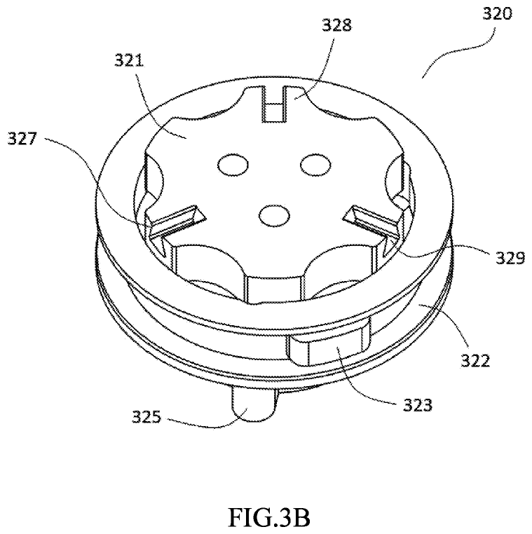

Referring to FIG. 3A and FIG. 3B, there are shown schematic views of a cord-receiving device according to a specific embodiment of the present disclosure. FIG. 3A shows the top side of the cord-receiving device. FIG. 3B shows the bottom side of the cord-receiving device. In the embodiment illustrated by FIG. 3A and FIG. 3B, a cord-receiving device 320 comprises a first cord-receiving portion 321 and a second cord-receiving portion 322. The second cord-receiving portion 322 is pivotally rotatable relative to the first cord-receiving portion 321 while being connected to the first cord-receiving portion 321 (to constitute a cord-receiving mechanism of the cord-receiving device 320). Thus, the second cord-receiving portion 322 rotates pivotally about an axis 320A relative to the first cord-receiving portion 321. In a specific embodiment, after the fixing cord has come into contact with the fixing devices and has been fixed to the second cord-receiving portion 322, healthcare workers rotate pivotally the second cord-receiving portion 322 in a second direction 820 relative to the first cord-receiving portion 321 to reduce the exposed length of the fixing cord, draw the fixing devices closer to each other, and/or increase the tension of the fixing cord. Then, the healthcare workers rotate pivotally the second cord-receiving portion 322 in the second direction 820 relative to the first cord-receiving portion 321 at any time and as needed to further reduce the exposed length of the fixing cord or rotate pivotally the second cord-receiving portion 322 in a first direction 810 relative to the first cord-receiving portion 321 at any time and as needed to increase the exposed length of the fixing cord, draw the fixing devices away from each other, and/or reduce the tension of the fixing cord.

In the embodiment illustrated by FIG. 3A and FIG. 3B, the first cord-receiving portion 321 has second joining parts 327, 328, 329. The first cord-receiving portion 321 is connected to a first joining part of the first fixing device by one of the second joining parts 327, 328, 329. The second cord-receiving portion 322 has a third auxiliary fixing portion 323 and a grip portion 325. The fixing cord is fixed to the third auxiliary fixing portion 323 of the cord-receiving device 320. The grip portion 325 is gripped by a user to operate the second cord-receiving portion 322 in order for the second cord-receiving portion 322 to rotate pivotally relative to the first cord-receiving portion 321. In a specific embodiment, the first joining part is a dovetail male socket, and the second joining parts 327, 328, 329 are dovetail sockets.

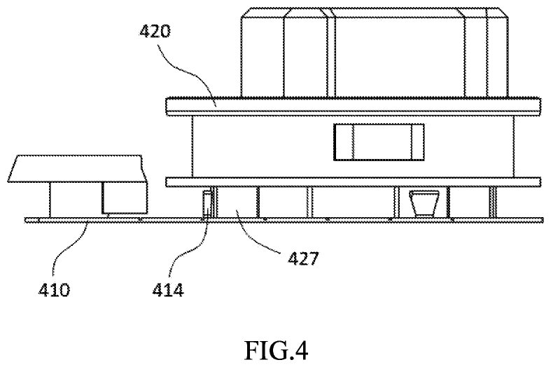

Referring to FIG. 4, there is shown a schematic view of a cord-receiving device connected to the first fixing device according to a specific embodiment of the present disclosure. In the embodiment illustrated by FIG. 4, a first joining part 414 of a first fixing device 410 is a dovetail male socket, and a second joining part 427 of a cord-receiving device 420 is a dovetail sockets. The first joining part 414 of the first fixing device 410 is connected to the second joining part 427 of the cord-receiving device 420. Preferably, the first joining part 414 of the first fixing device 410 is unfastenably connected to the second joining part 427 of the cord-receiving device 420. The first joining part and the second joining part are not necessarily a dovetail male socket and a dovetail sockets, respectively; instead, the first joining part and the second joining part may take on any structures or shapes conducive to interconnection as needed. Furthermore, the first joining part and the second joining part are not necessarily connected in the way shown in FIG. 4; instead, the first joining part and the second joining part may be connected in any other ways, for example, by engagement, adhesion and stapling, but the present disclosure is not limited thereto.

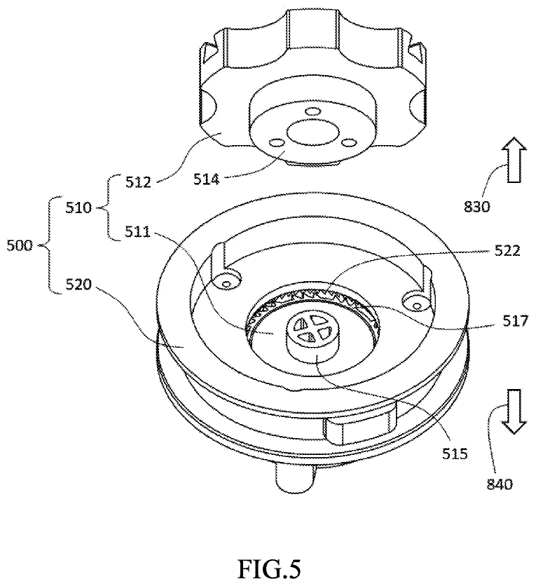

Referring to FIG. 5, there is shown a schematic view of a cord-receiving device according to a specific embodiment of the present disclosure. In the embodiment illustrated by FIG. 5, a cord-receiving device 500 comprises a first cord-receiving portion 510 and a second cord-receiving portion 520. The first cord-receiving portion 510 comprises an upper segment portion 511 and a lower segment portion 512. The upper segment portion 511 of the first cord-receiving portion 510 has a first aiming portion 515. The lower segment portion 512 of the first cord-receiving portion 510 has a second aiming portion 514. When the upper segment portion 511 is connected to the lower segment portion 512 to form the first cord-receiving portion 510, the first aiming portion 515 of the upper segment portion 511 is aimed at and connected to the second aiming portion 514 of the lower segment portion 512. The upper segment portion 511 of the first cord-receiving portion 510 is disposed inside of the second cord-receiving portion 520. The lower segment portion 512 of the first cord-receiving portion 510 is disposed outside of the second cord-receiving portion 520. Preferably, the upper segment portion 511 and the lower segment portion 512 of the first cord-receiving portion 510 move relative to the second cord-receiving portion 520 (for example, upward or downward) or rotate pivotally; thus, the first cord-receiving portion 510 moves or rotates pivotally relative to the second cord-receiving portion 520.

In the embodiment illustrated by FIG. 5, the upper segment portion 511 of the first cord-receiving portion 510 has a first engaging portion 517, whereas the second cord-receiving portion 520 has a second engaging portion 522. The first engaging portion 517 and the second engaging portion 522 are engaged with each other. Preferably, the first engaging portion 517 and the second engaging portion 522 are separably engaged with each other. For example, when the cord-receiving device 500 is in a first state, the first engaging portion 517 and the second engaging portion 522 are engaged with each other in order for the first cord-receiving portion 510 to be fixed to the second cord-receiving portion 520 (to prevent the second cord-receiving portion 520 from pivotally rotating relative to the first cord-receiving portion 510). When the cord-receiving device 500 is in a second state, the first engaging portion 517 and the second engaging portion 522 are separated, so that the second cord-receiving portion 520 rotates pivotally relative to the first cord-receiving portion 510.

In a specific embodiment, the cord-receiving device 500 comprises a resilient mountings disposed between the first cord-receiving portion 510 and the second cord-receiving portion 520. The resilient mountings is in contact with the first cord-receiving portion 510 and the second cord-receiving portion 520. The resilient mountings is disposed between the upper segment portion 511 of the first cord-receiving portion 510 and the second cord-receiving portion 520. The resilient mountings is in contact with the upper segment portion 511 of the first cord-receiving portion 510 and the second cord-receiving portion 520. When the resilient mountings is in a stretched state, the resilient mountings moves the upper segment portion 511 of the first cord-receiving portion 510 and the second cord-receiving portion 520 to not only allow the second cord-receiving portion 520 to move in a direction 840 and away from the first cord-receiving portion 510 (i.e., allowing the second cord-receiving portion 520 to move away from the lower segment portion 512 of the first cord-receiving portion 510) but also allow the first engaging portion 517 and the second engaging portion 522 to be engaged with each other, thereby causing the cord-receiving device 500 to be in the first state. When the resilient mountings is in a compressed state, not only does the second cord-receiving portion 520 move in a direction 830 relative to the first cord-receiving portion 510 (i.e., toward the lower segment portion 512 of the first cord-receiving portion 510), but the first engaging portion 517 and the second engaging portion 522 are separated, thereby allowing the cord-receiving device 500 to be in the second state. In a specific embodiment, the resilient mountings is normally in the stretched state, but the resilient mountings is in the compressed state under an applied force (for example, when a user presses the second cord-receiving portion 520 downward to cause the second cord-receiving portion 520 to move toward the lower segment portion 512 of the first cord-receiving portion 510.) The resilient mountings is, for example, a spring, but the present disclosure is not limited thereto.

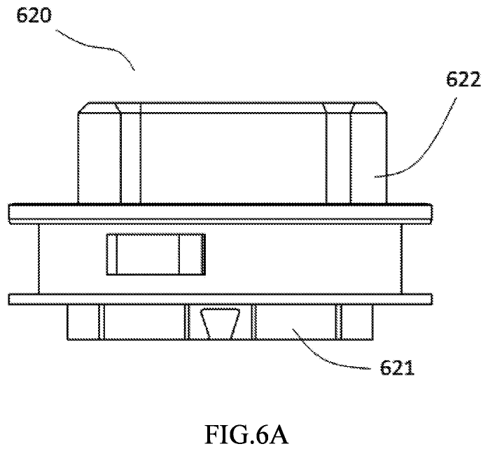

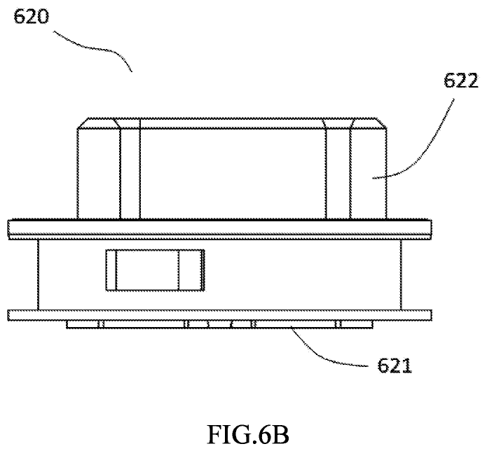

Referring to FIG. 6A and FIG. 6B, there are shown schematic views of a cord-receiving device according to a specific embodiment of the present disclosure. In the embodiment illustrated by FIG. 6A, when a second cord-receiving portion 622 is not subjected to any pressing force, the resilient mountings is in the stretched state, and a cord-receiving device 620 is in the first state, preventing the second cord-receiving portion 622 from rotating pivotally relative to a first cord-receiving portion 621. In the embodiment illustrated by FIG. 6B, when the second cord-receiving portion 622 is subjected to a pressing force, the resilient mountings is in the compressed state, and the cord-receiving device 620 is in the second state, allowing the second cord-receiving portion 622 to move toward the first cord-receiving portion 621 and rotate pivotally relative to the first cord-receiving portion 621.

In a specific embodiment, a cord-receiving device of the medical fixing system of the present disclosure comprises a first sensing portion and an warning signal. The first sensing portion senses the tension of the fixing cord (i.e., cord tension). The first sensing portion generates an data alert when the tension is less than a predetermined tension threshold (known as a first tension threshold), and the warning signal sends an warning signal according to the data alert. Thus, the warning signal sends the warning signal when the medical fixing system fails to apply an acting force great enough to draw the skin surrounding the target lesion closer to each other for any reasons (for example, when the fixing devices loosen, but the present disclosure is not limited thereto.) In a specific embodiment, the first sensing portion generates an data alert when the tension is greater than a predetermined tension threshold (known as a second tension threshold), and the warning signal sends an warning signal according to the data alert. The first sensing portion is, for example, a pressure sensor, but the present disclosure is not limited thereto. In a specific embodiment, the first sensing portion generates a tension data according to the tension of the fixing cord and sends the data alert and/or the tension data to a data-receiving device when the tension is less than a predetermined first tension threshold or greater than a predetermined second tension threshold. The data-receiving device is, for example, a cellphone, computer, server, and medical device, but the present disclosure is not limited thereto. The warning signal is, for example, a sound message or a flash message, but the present disclosure is not limited thereto.

In a specific embodiment, a cord-receiving device of the medical fixing system of the present disclosure comprises a second sensing portion and an warning signal. The second sensing portion senses the distance between the cord-receiving device and the second fixing device (for example, between one of the second fixing devices and the second sensing portion). The second sensing portion generates an data alert when the distance is less than a predetermined distance threshold (known as a first distance threshold), and the warning signal sends an warning signal according to the data alert. In a specific embodiment, the second sensing portion generates an data alert when the distance is greater than a predetermined distance threshold (known as a second distance threshold), and the warning signal sends an warning signal according to the data alert. The second sensing portion is, for example, an ultrasonic sensor or a photoelectric sensor, but the present disclosure is not limited thereto. In a specific embodiment, the second sensing portion generates a distance data according to the distance between a cord-receiving device and a second fixing device, and the second sensing portion sends the data alert and/or the distance data to a data-receiving device when the distance is less than a predetermined first distance threshold or greater than a predetermined second distance threshold. The data-receiving device is, for example, a cellphone, computer, server, and medical device, but the present disclosure is not limited thereto. The warning signal is, for example, a sound message or a flash message, but the present disclosure is not limited thereto.

A medical fixing system of the present disclosure is described above and illustrated by drawings. Specific embodiments of the present disclosure serve an illustrative purpose only. Various changes may be made to the embodiments of the present disclosure without departing from the spirit and claims of the present disclosure and must be deemed falling within the scope of the claims of the present disclosure. Thus, the specific embodiments described in this specification are not restrictive of the present disclosure. Accordingly, the real scope and spirit of the present disclosure should be defined by the appended claims.

What is claimed is:

1. A medical fixing system, comprising:
a first fixing device fixed to a target lesion and having a first joining part and a first auxiliary fixing portion;
a second fixing device fixed to the target lesion and having a second auxiliary fixing portion;
a cord-receiving device connected to the first joining part of the first fixing device; and
a fixing cord in contact with the first auxiliary fixing portion and the second auxiliary fixing portion and connected to the cord-receiving device,
wherein the cord-receiving device has a cord-receiving mechanism and adjusts an exposed length of the fixing cord with the cord-receiving mechanism.

2. The medical fixing system of claim 1, wherein the first fixing device has a first adhesive side and is fixed to the target lesion through the first adhesive side, and the second fixing device has a second adhesive side and is fixed to the target lesion through the second adhesive side.

3. The medical fixing system of claim 1, wherein the cord-receiving device comprises:
a first cord-receiving portion having a second joining part and connected to the first joining part of the first fixing device by the second joining part; and
a second cord-receiving portion, with the fixing cord being fixed to the second cord-receiving portion,
wherein the cord-receiving mechanism is the second cord-receiving portion's being pivotally rotatable relative to the first cord-receiving portion while being connected to the first cord-receiving portion.

4. The medical fixing system of claim 3, wherein the exposed length of the fixing cord increases gradually when the second cord-receiving portion rotates pivotally in a first direction relative to the first cord-receiving portion, wherein the exposed length of the fixing cord decreases gradually when the second cord-receiving portion rotates pivotally in a second direction relative to the first cord-receiving portion.

5. The medical fixing system of claim 3, wherein the first joining part is a dovetail male socket, and the second joining part is a dovetail sockets.

6. The medical fixing system of claim 3, wherein the second cord-receiving portion has a third auxiliary fixing portion, and the fixing cord is fixed to the third auxiliary fixing portion of the cord-receiving device.

7. The medical fixing system of claim 3, wherein the first cord-receiving portion has a first engaging portion, and the second cord-receiving portion has a second engaging portion, wherein the first engaging portion and the second engaging portion are engaged with each other when the cord-receiving device is in a first state, allowing the first cord-receiving portion to be fixed to the second cord-receiving portion, wherein the first engaging portion and the second engaging portion are separated from each other when the cord-receiving device is in a second state, allowing the second cord-receiving portion to rotate pivotally relative to the first cord-receiving portion.

8. The medical fixing system of claim 7, wherein the cord-receiving device comprises a resilient mountings disposed between the first cord-receiving portion and the second cord-receiving portion and in contact with the first cord-receiving portion and the second cord-receiving portion, wherein the cord-receiving device is in the first state when the resilient mountings is in a stretched state, wherein the cord-receiving device is in the second state when the resilient mountings is in a compressed state.

9. The medical fixing system of claim 1, wherein the cord-receiving device comprises a first sensing portion and an warning signal, wherein the first sensing portion senses a tension of the fixing cord and generates an data alert when the tension is less than a first tension threshold or greater than a second tension threshold, wherein the warning signal sends an warning signal according to the data alert.

10. The medical fixing system of claim 9, wherein the first sensing portion generates a tension data according to the tension of the fixing cord and sends the data alert and/or the tension data to a data-receiving device when the tension is less than the first tension threshold or greater than the second tension threshold.

11. The medical fixing system of claim 1, wherein the cord-receiving device comprises a second sensing portion and an warning signal, wherein the second sensing portion senses a distance between the cord-receiving device and the second fixing device and generates an data alert when the distance is less than a first distance threshold or greater than a second distance threshold, wherein the warning signal sends an warning signal according to the data alert.

12. The medical fixing system of claim 11, wherein the second sensing portion generates a distance data according to the distance between the cord-receiving device and the second fixing device and sends the data alert and/or the distance data to a data-receiving device when the distance is less than the first distance threshold or greater than the second distance threshold.

13. The medical fixing system of claim 1, wherein the fixing cord is one selected from medical sutures, elastic bands or wires.

14. A medical fixing system, capable of adjusting an exposed length of a fixing cord at a target lesion, the medical fixing system comprising:
a first fixing device fixed to the target lesion and having a first joining part and a first auxiliary fixing portion;
a second fixing device fixed to the target lesion and having a second auxiliary fixing portion; and
a cord-receiving device connected to the first joining part of the first fixing device,
wherein the fixing cord is connected to the cord-receiving device and in contact with the first auxiliary fixing portion and the second auxiliary fixing portion,
wherein the cord-receiving device has a cord-receiving mechanism and adjusts the exposed length of the fixing cord with the cord-receiving mechanism to not only allow the fixing cord to drive the first fixing device and the second fixing device but also allow the first fixing device and the second fixing device to be drawn closer to or away from each other.

15. The medical fixing system of claim 14, wherein the cord-receiving device comprises:
a first cord-receiving portion having a second joining part and connected to the first joining part of the first fixing device by the second joining part; and
a second cord-receiving portion, with the fixing cord being fixed to the second cord-receiving portion,
wherein the cord-receiving mechanism is the second cord-receiving portion's being pivotally rotatable relative to the first cord-receiving portion while being connected to the first cord-receiving portion.

16. The medical fixing system of claim 15, wherein the first cord-receiving portion has a first engaging portion, and the second cord-receiving portion has a second engaging portion, wherein the first engaging portion and the second engaging portion are engaged with each other when the cord-receiving device is in a first state, allowing the first cord-receiving portion to be fixed to the second cord-receiving portion, wherein the first engaging portion and the second engaging portion are separated from each other when the cord-receiving device is in a second state, allowing the second cord-receiving portion to rotate pivotally relative to the first cord-receiving portion.

17. The medical fixing system of claim 16, wherein the cord-receiving device comprises a resilient mountings disposed between the first cord-receiving portion and the second cord-receiving portion and adapted to be in contact with the first cord-receiving portion and the second cord-receiving portion, wherein the cord-receiving device is in the first state when the resilient mountings is in a stretched state, wherein the cord-receiving device is in the second state when the resilient mountings is in a compressed state.
